(19) United States
(12) Patent Application Publication
(10) Pub. No.: US 2006/0264805 A1
(43) Pub. Date: Nov. 23, 2006

(54) Permeabilization of biological membranes

(75) Inventors: Parminder Singh, Union City, CA (US); Gary W. Cleary, Los Altos Hills, CA (US)

Correspondence Address: MINTZ, LEVIN, COHN, FERRIS, GLOVSKY AND POPEO, P.C, 1400 PAGE MILL ROAD, PALO ALTO, CA, 94304-1124, US

(73) Assignee: Corium International, Inc., Redwood City, CA (US)

(21) Appl. No.: 11/433,664

(22) Filed: May 11, 2006

Related U.S. Application Data
(60) Provisional application No. 60/680,399, filed on May 11, 2005.

(51) Int. Cl. A61N 1/30

(52) U.S. Cl. 604/20

(57) ABSTRACT

In one embodiment of the invention, electrodes are brought into the vicinity of a biological membrane such as human skin. Through those electrodes, a current is driven. As a result of that current, at one electrode there occurs a reaction in which the pH of the water or other fluid in a small area of the membrane is lowered or raised. The lowered or raised pH causes substances present in the membrane to denature or decompose, resulting in the formation of a channel in the membrane. In another embodiment of the invention, a high volatility fluid is applied to a membrane from the outside. A small area of the membrane is heated. The high volatility fluid expands and then vaporizes. As a result of the expansion and vaporization of the fluid, a channel is created in the membrane.

### CROSS-REFERENCE TO RELATED APPLICATIONS

This application claims priority under 35 U.S.C. § 119(e)(1) to U.S. Provisional Patent Application No. 60/680,399, filed May 11, 2005, which is incorporated herein by reference in its entirety.

### TECHNICAL FIELD

The technical field of the invention is the movement of substances across biological membranes such as human skin, and specifically techniques which serve to increase the permeability of such membranes.

### BACKGROUND

Many drugs are unable to penetrate the human skin at therapeutically useful rates. It has therefore long been a goal to artificially enhance the permeability of human skin so as to allow such drugs to be administered transdermally. Such enhancement of permeability can also in some cases be useful in allowing removal of bodily fluids for analysis, e.g., glucose monitoring, and may be useful to enhance passage of materials through other types of biological membranes, such as those in the buccal cavity.

A useful survey of permeation enhancement techniques is found in Percutaneous Permeation Enhancers (Eric W. Smith & Howard I. Maibach eds., 1995). A wide variety of chemical substances have been proposed as permeation enhancers. Some are detailed in the Smith-Maibach book just cited; many others may be found in the patent literature. The use of ultrasound and electric fields has also been proposed. Iontophoresis, where the electric field imparts a drift velocity to ions, has been studied. Electroporation, where a pulsed electric field temporarily causes enhanced permeability, has also been the object of considerable research.

The barrier to skin permeation is believed to lie primarily in the outermost layer of skin, the stratum corneum, which contains dead cells called “keratinocytes” filled with the fibrous protein keratin, lipids in the extracellular space, and typically about 15-20% water. The lipids are believed to be about 50% ceramides, 25% cholesterol, 15% free fatty acids, and 5% cholesterol sulfate, and are thought to form bilayers. See in this regard Mechanisms of Transdermal Drug Delivery (Russell O. Potts & Richard H. Guy eds., 1997), which contains discussions of the stratum corneum and its constituents. Other biological membranes also comprise keratin to a greater or lesser degree, for example the keratinized epithelium, which is found for example in the mouth and lips, and the so-called “parakeratinized” epithelium.

It has been proposed that needles of small diameter penetrating the stratum corneum would overcome its barrier to permeation by creating small channels through it. Drugs and biological fluids would be expected to move more easily through these small channels than through unmodified stratum corneum. Such needles would need to be only on the order of 20 micrometers long to penetrate the stratum corneum in many areas of the human skin. They can be fabricated by a variety of techniques described in the literature. See in this regard, for example, U.S. Pat. No. 6,451,240 issued to Procter & Gamble.

Another class of proposals for permeation enhancement has involved the destroying of small areas of the stratum corneum as a way to create small channels in the stratum corneum. The general idea of creating such channels is often referred to as “microporation.” One way that has been proposed to accomplish microporation is simply to contact the skin with small heated objects for a defined period of time and to rely on conduction to transfer the heat through the stratum corneum. It has been claimed that through careful design, the temperature of the heated objects can be set at a value and duration which causes living, innervated portions of the skin to experience no more than 45° C., and yet causes the stratum corneum immediately adjacent to those objects to reach temperatures above 100° C. See in this regard U.S. Pat. No. 6,142,939 to Altea and SpectRx.

Another way that has been proposed to burn channels in the stratum corneum is to use radiofrequency energy after the manner of radiofrequency tumor ablation, a well known technology. The radiofrequency energy is said to cause water molecules in an oblong area to oscillate, thereby heating the area frictionally and destroying the proteins in that area. See in this regard U.S. Pat. No. 6,711,435 to Transpharma.

The different techniques for achieving microporation which have been proposed have not yet reached regulatory approval as drug delivery mechanisms. There is a need for alternative techniques to form channels in skin and other biological membranes, which may have a superior side effect profile, better controllability, greater localization, or other advantages.

### SUMMARY OF THE INVENTION

In one embodiment of the invention, electrodes are brought into the vicinity of a biological membrane. Through those electrodes, a constant or time-varying current is driven. As a result of that current, at one electrode there occurs a reaction in which the pH of the water or other fluid in a small area of the biological membrane is either lowered or increased. The lowered or increased pH results in the formation of a channel in the membrane.

In another embodiment of the invention, a plurality of electrodes is provided suitable for applying electrical current to a biological membrane, together with an electronic system serving to energize the electrodes in a controlled manner. The electronic system is programmed, preferably digitally, to apply electrical current to some or all of the electrodes in such a manner as to reduce or increase the pH of the biological membrane.

In another embodiment of the invention, a high volatility fluid is applied to a biological membrane from the outside. A small area of the membrane is heated. The high volatility fluid expands and then vaporizes. As a result of the expansion and vaporization of the fluid, a channel is created in the membrane.

### DRAWINGS

FIG. 1 depicts a schematic version of electrodes and an electronic controller according to an embodiment of the invention.

### DETAILED DESCRIPTION OF EMBODIMENTS OF THE INVENTION

Before describing the present invention in detail, it is to be understood that this invention is not limited to specific reagents, materials, or device structures, as such may vary. It is also to be understood that the terminology used herein is for the purpose of describing particular embodiments only, and is not intended to be limiting.

It must be noted that, as used in this specification and the appended claims, the singular forms “a,” “an,” and “the” include both singular and plural referents unless the context clearly dictates otherwise. Thus, for example, reference to “an electrode” includes a plurality of electrodes as well as a single electrode, reference to “a channel” includes a plurality of channels as well as single channel, and the like.

By “transdermal” delivery, applicants intend to include both transdermal (or “percutaneous”) and transmucosal administration, i.e., delivery by passage of a drug through the skin or mucosal tissue. Transdermal administration may be intended to have a local (topical) effect or an effect on bodily organs or tissues further away from the site of administration (e.g., a systemic effect) or both.

In one embodiment of the invention, electrodes are brought into the vicinity of a biological membrane such as the epidermis. Through those electrodes, a constant or time-varying current is driven. As a result of that current, at one electrode there occurs a reaction in which the pH of the water or other fluid in a small area of the membrane is lowered or increased. The lowered or increased pH results in the formation of a channel in the membrane.

An example of a reaction which would lower the pH of water in a biological membrane would be an electrolytic reaction affecting water itself, such as 2H₂O→O₂+4H⁺+4e⁻. Here H⁺ denotes a hydronium ion, which is believed to exist in water in a hydrated state more accurately written as H⁺·H₂O or H₃O⁺ but conventionally written as H⁺. The electrons e⁻ generated by this electrolytic reaction would be taken up by the electrode by an electromotive force externally supplied. It is seen that, if this reaction takes place at an electrode, a hydronium ion is generated for each electron e⁻ which is taken up by the electrode. In this way, one may calculate the number of hydronium ions generated as a function of the current passing through the electrode.

An example of such a calculation is as follows. Suppose that it is desired to lower pH to 2 in a volume of 0.1 μL of water immediately surrounding a number of electrodes over a surface area of skin. The volume of 0.1 μL represents very roughly one third of the expected volume of water in 1 cm² of intact human skin under typical conditions (estimated from a stratum corneum thickness of 15 μm times 1 cm² times 20%, which is a rough estimate of the percentage water content of the stratum corneum). To generate x H⁺ ions we need an integrated current of x electronic charges. An electronic charge is e=1.6×10⁻¹⁹ C. To achieve a pH of 2 it is necessary to be 10⁻² molar in H⁺ ions. In a volume of 0.1 μL=10⁻⁷ L, we thus need 10⁻⁷×10⁻² moles, i.e., 10⁻⁹N_{A}, H⁺ ions, N_{A} being Avogadro's number. The integrated current we need is therefore 10⁻⁹N_{A}e=9.6×10⁻⁵ C. An ampere is a Coulomb per second, so that in one second one can create 0.01M H⁺ in 0.1 μL if one sends a current of 96 micromperes. Currents of this small value are commonly generated without harm in other types of diagnostic tests or treatments, for example in skin conductivity measurements.

It is also possible for there to occur a reaction which generates a hydroxide ion OH⁻, for example 2H₂O+2e⁻→2OH⁻+H₂. Such a reaction would tend to depress the concentration of hydronium ion on account of the equilibrium H⁺+OH⁻<−>H₂O, and thus would tend to raise the pH of the water in a biological membrane. Using the known equilibrium constant of the latter reaction, it is possible to calculate the electron current necessary to raise the pH to a particular level, for example 12.

In a single system of the invention, there may be some electrodes which raise pH and others which lower pH, for example by the electrolysis reactions given above. Conveniently, for example, electrodes at which electrons enter the biological membrane would raise pH and those at which electrons leave the biological membrane would lower pH.

Because it is desired to create small pores in a biological membrane, it is preferred that the area of contact of the electrodes with the membrane be small, preferably less than 100 μm in diameter, more preferably less than 10 μm in diameter or less than 5 μm in diameter. Preferably one electrode will generate one pore in the membrane. The electrodes may sit atop biological membrane or they may penetrate some distance into it. The electrodes are preferably integrated into an applicator which provides physical support for them and connects them to an electronic controller. The electrodes are preferably made of a conductive material at which a suitable electrochemical reaction, such as 2H₂O→O₂+4H⁺+4e⁻, will occur. An exemplary material is platinum. The electrodes may be formed, for example, as a plated or coated layer atop a supporting layer, using for example technologies of the kind employed in printed circuit board manufacturing. The electrodes may optionally be coated, for example, with a thin layer of a suitable acid or alkali. The electrodes or their housing are preferably supplied with a device for holding the electrodes firmly against a desired area of the biological membrane, for example the patient's skin. This device may be, for example, a strap made of a suitably elastic material.

As a consequence of the lowered or increased pH, channels may be formed in the biological membrane. In the case where this membrane is the human skin, it is believed that the proteins making up the cell surfaces of the keratinocytes and the keratin inside those cells will denature and/or decompose on account of the pH. This will create porosity which allows the passage of desired drugs in a manner analogous to porosity created by other techniques. Similar denaturation phenomena would be expected to occur in other biological membranes, for example in keratinized and parakeratinized epithelium. The lowered pH will eventually return to normal values as the excess H⁺ ions react or diffuse away or the treated surface is neutralized in some way. Similarly if pH is raised the pH should eventually return to normal values. It should be noted that the normal pH of the stratum corneum has been reported to lie between 4 and 5, so the skin is accustomed to a relatively high loading of H⁺ ions.

In addition to the electrodes at which the desired H⁺ or OH⁻ generating electrochemical reaction occurs, there may optionally be at least one electrode at which a current loop is closed without an electrochemical reaction having a major effect on pH occurring in the vicinity of the electrode. This current-loop-closing electrode may be a single electrode attached to any convenient portion of the patient's body, or a plurality of such electrodes. The current-loop-closing electrode or plurality of electrodes may be held by the housing which holds the electrodes at which the pH modifying reaction occurs or by a different housing. For example, the current-loop-closing electrodes may alternate with the electrodes at which the pH lowering/increasing reaction occurs within an array of electrodes, or alternatively they may surround such an array or comprise a set of gridlines within such an array. The current-loop-closing electrode or plurality of electrodes may be attached to a patient's skin or other biological membrane with a second device to hold them against the membrane, such as another strap. Optionally conductive jelly, for example of the type used for electrocardiography, may be employed. In some system configurations, rather than having a current-loop-closing electrode of the type just described, the system design is such that all electrodes can have a significant effect on pH at some point or in some modality of the system's operation.

The current flowing between the electrodes is preferably supplied by an electronic controller of some type. The controller may control the voltage between the electrodes in order to cause current to flow between them. The electronic controller may be battery operated or operate from mains power through a suitable electronic power supply producing DC/AC power. The electronic controller may employ or be connected to a digital computing device of some sort, such as a microprocessor, microcontroller, or application-specific integrated circuit (ASIC). Some or all of the activities performed by the electronic controller may be carried out under the control of computer programs, whether in the form of software or firmware.

The electronic controller is preferably designed to supply a settable quantity of integrated charge at an approximately constant current. As will be understood by those of skill in the art, supplying charge at a constant current will in general require the controller to vary the voltage between the electrodes if the resistance between the electrodes varies, for example, as a result of changes in the stratum corneum or other biological membrane. The design of constant current sources for low currents is known in the art. Reference may be made, for example, to Paul Horowitz & Winfield Hill, The Art of Electronics (2d ed. 1989). Alternatively, the current or voltage supplied at the electrodes could be varied over time according to a predetermined or adaptively determined waveform. The electronic controller could, for example, sense a decrease in the skin resistance which is being encountered, take that decrease as a sign that sufficient ablation of the stratum corneum has occurred, and cut back or end the supply of current to the particular electrode which is sensing that decrease in resistance and/or to other electrodes.

The selection of the integrated charge to be applied, of the speed at which it is applied, and/or of other parameters of the current applied may be made by pushbuttons, dials, or the like in a housing of the electronic controller or in a separate unit, or it may be made through a computer program which may print or automatically record on magnetic or other non-volatile storage the integrated charge applied and/or other parameters of the treatment. The printing and/or recording may be for medical recordkeeping or other purposes. Communication between the electronic controller and a separate unit or a computer running a computer program may be made, for example, through a suitable connector allowing data communication or through a wireless network. The electronic controller preferably complies with the UL 2601-1 standard or a similar standard for medical electronic equipment. Preferably there is a light such as a light emitting diode which indicates when the controller unit is active and supplying current to the patient's skin; alternatively, a buzzer or other indicator may be used for this purpose.

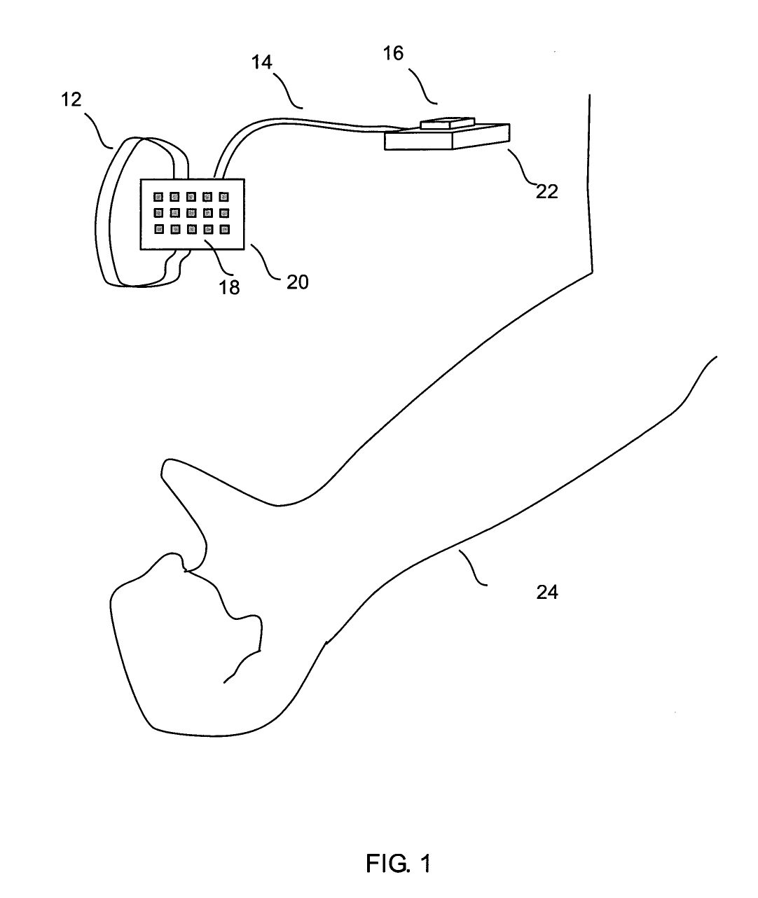

FIG. 1 schematically depicts an embodiment of the invention intended for use with human skin. Electrodes 18 are attached to a housing 20. Preferably the electrodes are more numerous and smaller than is depicted in the FIGURE. A strap 12 is used to hold the housing 20, and thus the electrodes 18, in contact with the skin. A controller is located in a different housing 22 and is connected through a connector 14, for example a ribbon connector comprising wires, to the housing 20, allowing the controller to energize the electrodes 18. A further electrode (not shown) is found on the underside of housing 22. A button 16 is used to cause the controller in housing 22 to cause a flow of current through electrodes 18. In use, both of the housings 20 and 22 are placed in contact with the skin of a patient 24, and then the button 16 is pressed to cause the current flow. The flow of electrons exits the skin and enters electrodes 18. This flow passes through suitable circuitry inside housing 20, through wires in connector 14, through the controller inside housing 22, and through the electrode on the underside of housing 22 back into the skin. In this embodiment no mechanism is shown for holding housing 22 in place against the skin, but a strap similar to 12 or some other mechanism could be added. Instead of or in addition to button 16, other more sophisticated user-actuated controls could be employed, or the entire controller could be commanded remotely, for example via wireless communication, from a computer. The button 16 could also alternatively be recessed in the housing 22 to avoid the possibility of accidental actuation. The pressure on button 16 or its degree of depression could be sensed and used as an input to set parameters of the current waveform being applied through the electrodes 18.

Many other physical and electrical arrangements besides that shown in FIG. 1 are possible. It is possible to integrate all electrodes into a single housing. The controller may be located inside this housing or separately. In a further variant on FIG. 1, the controller could be housed in the same housing as electrodes 18, with electrode 22 being a simple electrode similar in form, for example, to an electrocardiogram lead. Embodiments which are used for membranes other than skin may require, for example, different mechanisms to hold the electrodes in place, as for example a clamp to hold the electrodes against the interior of the buccal cavity.

Because the electrodes at which the pH is raised or lowered will be in contact with abraded skin or another permeabilized biological membrane, it may be desirable for a structure containing those electrodes to be disposable, in which case the structure might be designed to be readily inserted into and removed from a housing. Alternatively, the ready removability of such a structure could allow it to be sterilized more readily for reuse, for example by autoclaving.

In certain embodiments of the invention a separate controllable current or voltage source (for example, on an integrated circuit such as an ASIC) may be employed for each electrode or pair of electrodes. In other embodiments a single controllable current or voltage source drives more than one electrode or pair of electrodes in parallel. In an exemplary embodiment, an ASIC containing the electronics for the current sources would be in communication with a microcontroller over a wire or set of wires used by the microcontroller to indicate to the ASIC the current or voltage currently to be driven on each electrode or group of electrodes. The same wires, or a different set of wires, would optionally be used by the ASIC to indicate to the microcontroller the current or voltage or resistance or similar quantity being perceived at each electrode or group of electrodes. Suitable software in the microcontroller, responsive also to user actions or to communications with a computer, would guide the time course of the voltage or current being applied to each electrode or group of electrodes.

In another embodiment of the invention, a high volatility fluid is applied to a biological membrane. A small area of the membrane is heated. The high volatility fluid expands and then vaporizes, partially or completely. As a result of the expansion and vaporization of the fluid, a channel is created in the membrane.

It has come to be believed that the ability of heat to ablate the stratum corneum arises because the heat is able to vaporize the water in the stratum corneum. The vaporization causes a large expansion in volume, and the expansion in volume physically destroys the integrity of the lipid bilayers, keratinocyte cell surfaces, and/or the keratin contents of the keratinocytes. The expansion of the water through heating acts in some ways analogously to a small explosion in terms of the structural damage it causes to the tightly compacted structural materials of the stratum corneum.

The direct application of heat by conduction to the stratum corneum has the disadvantage, naturally, that without careful control, the temperature of the living layers of skin lying underneath the stratum corneum may rise to undesirable levels. This rise in temperature may cause patient discomfort or even injury.

It is therefore desirable to find a way to ablate small areas of the stratum corneum, creating the desired permeation-enhancing porosity, while raising the temperature to a lower degree than would otherwise be necessary. This may be accomplished by applying a high volatility fluid to the skin from the outside. Heating of this fluid to its volatilization temperature, which is lower than the volatilization temperature of water, accomplishes the same volume expansion and structural damage which volatilization of water can accomplish, but at a lower temperature, resulting in safer operation.

The high volatility fluids which may be employed in the method described above may be any suitable non-toxic and biocompatible fluids having a volatilization temperature lower than that of water. These include, for example, cyclohexane, ethyl alcohol, ethyl ether, isopropanol, methyl acetate, acetonitrile, hexane, heptane, pentane, ethyl formate, 1,2-dimethoxyethane, t-butylmethyl ether, acetone, ethyl acetate, isopropyl acetate, methylethyl ketone, and chloroform.

It is well known than the stratum corneum may be loaded with an excess quantity of water, in some cases two or three times its dry weight. An excess loading of the stratum corneum with water may ensue, for example, simply by placing the skin under an occlusive dressing, thus preventing the normal evaporation of water through the skin. The stratum corneum is also able to be wetted with substantial quantities of other fluids compatible with its aqueous or lipid components or both.

The high volatility fluids employed in the method described above may be applied to the stratum corneum by any means known to those of skill in the art, such as swabbing or direct contact of skin with a mass of fluid. The application of the fluid is preferably rapid so as to achieve the desired loading quickly.

The application of heat to the stratum corneum following application of the high volatility fluid may follow by any method known to those of skill in the art which allows the appropriate degree of control of the heat applied. The heat may thus be applied, for example, by conduction or by means of radiofrequency fields. It may be applied, for example, by placing against the skin one or more conductive elements which are heated by passing through each element an electric current. The conductive elements are preferably less than 100 μm in diameter, more preferably less than 10 μm in diameter or less than 5 μm in diameter. There is preferably one conductive element for each pore or channel sought to be created in the stratum corneum. Preferably the conductive elements are of low heat capacity. The conductive elements are preferably integrated into an applicator which provides physical support for them and optionally connects them to an electronic controller. Optionally, a mechanism may be added for cooling the conductive elements, for example by means of an endothermic chemical reaction or by Peltier cooling, when the time period during which heat is to be applied ends, in order to limit the heating of the skin. A mechanism may also optionally be included for measuring the temperature which is being applied to the skin.

The elements which supply heat to the skin following application of the high volatility fluid may be in a housing. The housing is preferably attachable to the skin firmly but then readily removable so as to easily withdraw the heating elements from contact with the skin. The housing may provide a thermal mass into which the heat remaining in the conductive elements can readily diffuse upon termination of the heat application process. If the conductive elements are heated by passing an electric current through them or if they are used to radiate electromagnetic fields into the skin, then the housing may supply the electrical conductors through which this electric current or electromagnetic energy is supplied. The conductive elements may be arranged, for example, in an array. On account of their contact with abraded skin, it may be desirable for them to be disposable and/or designed to be readily inserted into and removed from their housing for disposal or sterilization.

The electronic controller which controls the application of the heat to the skin may be battery operated or operate from mains power through a suitable electronic power supply producing DC power. The exact design of the electronic controller will depend on the method used to apply heat, although in general terms it will bear some similarities to the controller used in the pH lowering embodiment of the invention, particularly as regards its mechanical design and user interface. The controller may be integrated into a housing for the heat-supplying elements or may be in a separate unit. The controller may supply current to the heat-supplying elements either in order to heat them or in order to cause them to radiate electromagnetic fields into the skin. The radiated electromagnetic fields are preferably in the range of 2 kHz to 500 kHz, more preferably in the range of 5 kHz to 100 kHz. The generation of electric power at these frequency ranges is well known to those of skill in the art. Such frequency ranges have been used, as indicated earlier, in radiofrequency tumor ablation.

The electronic controller for heat application preferably allows settability of parameters such as the temperature to be achieved, the time period of operation, or the electromagnetic energy to be supplied. The electronic controller also preferably records and/or communicates with a system which can record or print some or all parameters of the treatment applied. The electronic controller preferably complies with the UL 2601-1 standard or a similar standard for medical electronic equipment. Preferably there is a light such as a light emitting diode which indicates when the controller unit is active and supplying heat to the patient's skin.

In another embodiment of the invention, a heat sensitive material is brought into contact with a biological membrane in the vicinity of small heating elements, for example as a coating on those elements or as a thin film placed between those small heating elements and the biological membrane. The heating elements are brought to a temperature in a controlled manner such that the heat sensitive material changes and becomes more able to decompose the membrane, for example through denaturing of proteins. The heat sensitive material might, for example, become more acidic or basic with changing temperature.

When enhanced permeability is achieved by means of the invention, it may be expected to last for a few days until the stratum corneum or other membrane can repair itself. Thus, a conventional drug-containing transdermal patch may advantageously be used to deliver a desired active ingredient over those days, taking advantage of the enhanced permeability. Some deviations from conventional patch design may, however, be desirable, for example, because a conventional patch might contain excipients which would enter the body if the patch were applied in an area of enhanced permeability. The drug-containing transdermal patch and the electrodes may be integrated in a common housing, forming an integrated system for microporation and drug delivery. Alternatively, they may both form part of an element which is inserted into a suitable housing which is then placed against the skin or other membrane.

A wide variety of active ingredients which have not been successfully delivered transdermally may be supplied by means of the invention. One active ingredient whose transdermal delivery has long been sought is insulin. Delivery by means of the invention could also be carried out for a wide variety of other drugs of different therapeutic classes, including for example analgesic agents, anesthetic agents, antiarthritic agents, respiratory drugs, including antiasthmatic agents, anticancer agents, including antineoplastic drugs, anticholinergics, anticonvulsants, antidepressants, antidiabetic agents, antidiarrheals, antihelminthics, antihistamines, antihyperlipidemic agents, antihypertensive agents, anti-infective agents such as antibiotics and antiviral agents, antiinflammatory agents, antimigraine preparations, antinauseants, antineoplastic agents, antiparkinsonism drugs, antipruritics, antipsychotics, antipyretics, antispasmodics, antitubercular agents, antiulcer agents, antiviral agents, anxiolytics, appetite suppressants, attention deficit disorder (ADD) and attention deficit hyperactivity disorder (ADHD) drugs, cardiovascular preparations including calcium channel blockers, CNS agents, beta-blockers and antiarrhythmic agents, central nervous system stimulants, cough and cold preparations, including decongestants, diuretics, genetic materials, herbal remedies, hormonolytics, hypnotics, hypoglycemic agents, immunosuppressive agents, leukotriene inhibitors, mitotic inhibitors, muscle relaxants, narcotic antagonists, nutritional agents, such as vitamins, essential amino acids and fatty acids, ophthalmic drugs such as antiglaucoma agents, parasympatholytics, peptide drugs, psychostimulants, sedatives, steroids, sympathomimetics, tranquilizers, and vasodilators including general coronary, peripheral and cerebral.

The creation of channels as discussed above may also serve to withdraw bodily fluids, for example, for purposes of analysis. A particularly preferred bodily fluid application of enhanced permeation is the monitoring of glucose in diabetics through the skin, employing less invasive means than those currently employed for that purpose. This is an important application because glucose monitoring must be done frequently, often more than once per day. In applications of this type, it may be desirable to design the electrode or conductive element housing in such a way that pumping action can be exercised tending to withdraw the fluid from the body through the channels and to transport the fluid conveniently to a monitoring device, potentially within the same housing, where the fluid can be monitored, for example, by measuring its optical absorbance at one or more wavelengths. Where pumping action is used, it may be desired to design the housing in such a way as to conform tightly to the skin, for example by making the portions of the housing which contact the skin suitably flexible. A discussion of techniques of interstitial fluid monitoring is found in U.S. Pat. No. 6,591,124, assigned to Procter & Gamble.

The creation of channels as discussed above may be combined with other methods of facilitating the permeation of drugs through the skin, for example, the methods discussed in the book Percutaneous Permeation Enhancers cited above. For example, it would be possible to combine the techniques of the invention with chemical permeation enhancers. Suitable enhancers include, for example, the following: sulfoxides such as dimethylsulfoxide (DMSO) and decylmethylsulfoxide (C₁₀MSO); ethers such as diethylene glycol monoethyl ether (available commercially as Transcutol®) and diethylene glycol monomethyl ether; surfactants such as sodium laurate, sodium lauryl sulfate, cetyltrimethylammonium bromide, benzalkonium chloride, poloxamer (231, 182, 184), poly(oxyethylene) sorbitans, e.g., Tween® (20, 40, 60, 80) and lecithin (see, e.g., U.S. Pat. No. 4,783,450); pentadecalactone; methyl nicotinate; cholesterol; bile salts; fatty acids such as lauric acid, oleic acid and valeric acid; fatty acid esters such as isopropyl myristate, isopropyl palmitate, methylpropionate and ethyl oleate; polyols and esters thereof such as propylene glycol, propylene glycol monolaurate, ethylene glycol, glycerol, butanediol, polyethylene glycol and polyethylene glycol monolaurate (PEGML; see, e.g., U.S. Pat. No. 4,568,343); phospholipids such as phosphatidyl choline, phosphatidyl ethanolamine, dioleoylphosphatidyl choline (DOPC), dioleoylphosphatidyl glycerol (DOPG) and dioleoylphoshatidyl ethanolamine (DOPE); amides and other nitrogenous compounds such as urea, dimethylacetamide (DMA), dimethylformamide (DMF), 2-pyrrolidone, 1-methyl-2-pyrrolidone, ethanolamine, diethanolamine and triethanolamine; terpenes; alkanones; cyclodextrins and substituted cyclodextrins such as dimethyl-β-cyclodextrin, trimethyl-β-cyclodextrin and hydroxypropyl-β-cyclodextrin; and organic acids, particularly salicylic acid and salicylates, citric acid, and succinic acid. Particularly preferred permeation enhancers include hydroxypropyl-β-cyclodextrin, isopropyl myristate, oleic acid, pentadecalactone, propylene glycol, propylene glycol monolaurate and triethanolamine. It is also possible to combine the channel creation techniques described in this application with physical means of increasing the permeation of active substances through the skin, for example, iontophoresis for active substances which can be delivered as ions, electrotransport, electroporation, or ultrasound. It would also be possible to use two or more of these chemical enhancers or physical means of enhancing permeation in combination with the channels created in the manners described above. For iontophoretic delivery in particular it would be possible to design electrode systems which are suitable both for ablation through pH control as described above and also for iontophoretic delivery. The relation between these two modalities of operation would be primarily a question of the manner in which the programming in a controller commands that the electrodes be driven with voltage and/or current. The choice between pH control and iontophoretic transport of a desired active ingredient could be made by any of the means described above for the programming of the systems of the invention. The choice could be made for example in a separate computer or controller having a conventional user interface, or alternatively in the same unit that contains the electrodes applied to the skin, with a user interface comprising for example dials or pushbuttons.

It is to be understood that while the invention has been described in conjunction with the preferred specific embodiments thereof, that the foregoing description and the examples that follow are intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties. However, where a patent, patent application, or publication containing express definitions is incorporated by reference, those express definitions should be understood to apply to the incorporated patent, patent application, or publication in which they are found, and not to the remainder of the text of this application, in particular the claims of this application.

We claim:

1. A method of increasing the permeability of a biological membrane of a human or animal comprising:
(a) bringing an electrode into the vicinity of the biological membrane,
(b) driving a current through the electrode,
(c) at least in part as a result of the current, modifying the pH of a fluid in a portion of the biological membrane in the vicinity of the electrode,
(d) at least in part as a result of the modified pH, creating a channel in the biological membrane.

2. The method of claim 1, wherein step (a) comprises holding the electrode in place by means of a mechanical device.

3. The method of claim 1, wherein step (a) comprises contacting the biological membrane with the electrode.

4. The method of claim 3, wherein step (a) comprises penetrating the biological membrane with the electrode.

5. The method of claim 1, wherein the area of contact of the electrode with the biological membrane has a diameter no greater than about 50 micrometers.

6. The method of claim 1, wherein the direct current is chosen to achieve at least approximately a selected total quantity of charge.

7. The method of claim 1, wherein step (c) lowers the pH to below about 3.

8. The method of claim 1, wherein step (c) increases the pH to above about 9.

9. The method of claim 1, wherein the electrode is coated with a material which modifies pH.

10. A device for increasing the permeability of the skin of a biological membrane comprising:
(a) a plurality of electrodes suitable for applying electrical current to the membrane,
(b) an electronic system for energizing the electrodes in a controlled manner so as to cause a current to pass through selected ones of the plurality of electrodes, the current resulting in modifying the pH of a fluid in a portion of the membrane and, at least in part as a result of the modified pH, increasing the permeability of the membrane.

11. The device of claim 10, wherein at least one of the selected electrodes penetrates the membrane.

12. The device of claim 10, wherein the electronic system comprises or communicates with a digital electronic system.

13. The device of claim 10, wherein the electronic system is energized by mains power.

14. The device of claim 10, further comprising a housing for holding at least some of the selected electrodes.

15. The device of claim 10, further comprising a releasable device for keeping the electrodes in contact with the membrane.

16. A method of increasing the permeability of a biological membrane comprising:
(a) applying a biocompatible fluid having a boiling point lower than that of water to a portion of the membrane,
(b) heating an area of the membrane to a temperature at least about the vaporization point of the biocompatible fluid, thereby creating at least one channel in the membrane.

17. The method of claim 16, wherein the biocompatible fluid is chosen from the group consisting of cyclohexane, ethyl alcohol, ethyl ether, isoproanol, methyl acetate, acetonitrile, hexane, heptane, pentane, ethyl formate, 1,2-dimethoxyethane, t-butylmethyl ether, acetone, ethyl acetate, isopropyl acetate, methylethyl ketone, and chloroform.

18. The method of claim 16, wherein the heating of an area of the membrane results at least in part from placing a heated element in proximity to the membrane.

19. The method of claim 18, wherein the heated element is heated by passing an electric current through it.

20. The method of claim 16, wherein the heating of an area of the membrane results at least in part from providing an electromagnetic field of frequency at least 2 kHz which penetrates the membrane.

21. A method of increasing the permeability of a biological membrane, comprising:
(a) bringing a heat sensitive material into contact with the membrane in the vicinity of small heating elements,
(b) controlling the temperature of the small heating elements such that the heat sensitive material changes and becomes more able to decompose the biological membrane.

22. The method of claim 21, wherein the change in the heat sensitive material in step (b) is a change in pH.

23. The method of claim 21, wherein the change in the heat sensitive material in step (b) is a change in phase.
